(19) United States
(12) Patent Application Publication
(10) Pub. No.: US 2022/0390441 A1
(43) Pub. Date: Dec. 8, 2022

(54) ANALYTICAL MEDIUM FOR IMMUNOASSY AND METHOD FOR DETECTING ANALYTES IN SUCH A MEDIUM

(71) Applicant: ATWARE, Le Plessis-Robinson (FR)

(72) Inventor: Lionel Cima, Saint Cyr Sur Loire (FR)

(21) Appl. No.: 17/755,487

(22) PCT Filed: Sep. 9, 2020
(86) PCT No.: PCT/FR2020/051551

§ 371 Date: Apr. 29, 2022

(30) Foreign Application Priority Data
Oct. 31, 2019 (FR) 1912273

(51) Int. Cl. G01N 33/543; G01N 33/49

(52) U.S. Cl. CPC G01N 33/54326; G01N 33/492

(57) ABSTRACT

An analysis medium for immunoassay comprises a substrate for promoting the natural or forced flow of a liquid sample likely to comprise analytes, the substrate having a capture zone. The capture zone comprises, immobilized on or in the substrate, a plurality of first capture agents capable of selectively retaining complexes bound to the analytes and a plurality of second capture agents capable of selectively retaining reference agents. A method for detecting the presence of analytes in a liquid sample may be performed using this analysis medium.

### CROSS-REFERENCE TO RELATED APPLICATIONS

This application is a national phase entry under 35 U.S.C. § 371 of International Patent Application PCT/FR2020/051551, filed Sep. 9, 2020, designating the United States of America and published as International Patent Publication WO 2021/084170 A1 on May 6, 2021, which claims the benefit under Article 8 of the Patent Cooperation Treaty to French Patent Application Serial No. 1912273, filed Oct. 31, 2019.

### TECHNICAL FIELD

The present disclosure relates to the detection of the presence of analytes in a liquid sample, using magnetic microbeads. It also relates to an analysis medium for detecting analytes in the sample, which makes it possible to implement the method. The present disclosure is applicable in the field of immunoassay.

More generally, the present disclosure relates to a method for simultaneous measurement of respective masses of a first and of a second plurality of superparamagnetic nanoparticles, the nanoparticles of the first and of the second pluralities having different superparamagnetic features.

### BACKGROUND

In the field of immunoassay, and as is disclosed by, for example, the document by Huang, Zhen, et al. “Application and development of superparamagnetic nanoparticles in sample pretreatment and immunochromatographic assay.” TrAC Trends in Analytical Chemistry 114 (2019): 151-170, it is conventional to use an analysis medium that allows for detection of an analyte, or indeed measurement of its amount or its concentration, for example, by natural lateral flow of a liquid sample on a test strip, or by forced flow through a column comprising a porous material.

Thus, and with reference to FIGS. 1A and 1B, a test strip 1 of the prior art comprises a substrate formed of a material, for example, porous, which makes it possible, by capillarity, to promote the flow of the liquid sample E likely to contain the analytes A. The sample E is poured onto a receiving zone 1a for impregnating the substrate, and flows laterally from the receiving zone 1a of the sample 1. Magnetically marked agents 2 (comprising, for example, antibodies capable of binding to the analytes) are arranged in the region of the receiving zone 1a or in the vicinity of the zone, in order to dissolve in the sample E, to bind to the analytes A, and to form magnetically marked analyte-agent complexes.

The test strip 1 forming the analysis medium also comprises a capture zone 1c where immobilized capture agents 3 are located, which are typically the same antibodies as those contained in the magnetically marked agents 2. The complexes and the magnetically marked and non-bound agents 2 migrate from the receiving zone 1a toward the capture zone 1c, and pass through an intermediate migration zone 1b.

The complexes are selectively retained by the capture agents 3 in the region of the capture zone 1c. The magnetically marked agents 2 that are not bound to analytes A continue their migration beyond the zone. The detection of the magnetically marked agents 2 that are bound to analytes A in the capture zone 1c indirectly provides a means for detecting the presence of analytes A in the sample.

The test strip 1 also comprises a control zone 1d that is arranged downstream of the capture zone 1c in the flow direction of the sample E, and on which the control agents 4 remain, immobilized. These agents 4 are designed to bind to the magnetically marked agents 2 that are not bound to analytes A, in order to retain them in the control zone 1d.

The detection of the magnetically marked agents 2 in the control zone 1d provides means that make it possible to indicate that the test has been performed properly, i.e., that the sample E has indeed flowed from the receiving zone 1a as far as the capture zone 1c.

As has already been stated, the analysis medium can alternatively be formed of column filled with a porous material in which the capture agents 3 are grafted. In this embodiment of the immunoassay, the migration of the complexes and the magnetically marked and non-bound agents 2 is caused by forcing the flow of the sample E through the porous material of the column. It is interesting to note that, in this case, there is no possibility of forming a control zone.

Whatever the embodiment selected, i.e., a strip or a test column, the magnetically marked agents 2 are usually formed of magnetic microbeads of micrometric dimensions, comprising superparamagnetic nanoparticles. The microbeads are functionalized (for example, using antibodies) so as to bind to analytes A of the sample. The nanoparticles are of nanometric dimensions (i.e., the large diameter of which is between a few nanometers and a few tens of nanometers), for example, particles of Fe, Ni or Co, or other mixtures. The particles may be incorporated into the magnetic microbeads by way of a binder.

The superparamagnetic materials that are involved in forming the microbeads have the particular feature of having a non-linear magnetic cycle B(H) when the excitation magnetic field H varies over a sufficiently extended range. B denotes the magnetic induction in the material, caused by the field H. It also has the advantage of not having any remanence, i.e., B(0)=0.

In order to detect the presence or absence of analytes A in the sample, a measuring device is used that is capable of locating the presence of superparamagnetic nanoparticles, or indeed of estimating the present amount of nanoparticles. For this purpose, two successive measurements are performed—a first in the region of the capture zone 1c, a second in the region of the control zone 1d, when this exists. The result of these measurements makes it possible to verify that the immunological test has been performed properly (detection of a signal in the region of the control zone 1d), and to determine the presence or absence of analytes A in the analyzed sample (presence or absence of a signal in the region of the capture zone 1c).

In order to perform these measurements, it is possible to use a measuring device that makes use of the non-linearity of the magnetic cycle of the nanoparticles incorporated in the microbeads, for example, in accordance with that described in European Patent Application EP3314248. The device, in particular, makes it possible to estimate the amounts of superparamagnetic materials present in the control 1d and capture 1c zones, respectively, over the course of two separate measuring steps.

The device comprises four coils that are arranged in a measuring circuit, including one measuring coil in which the zone of the test strip 1 to be evaluated is placed.

The four coils are arranged electrically in series, and are identical to one another. High-frequency (HF), low-frequency (BF), and direct (DC) currents pass through the coils, according to a very precise configuration intended to develop a different magnetic field in each of the coils. The superparamagnetic material is exposed to a high-frequency and a low-frequency magnetic field, and, on account of the non-linear behavior of the material, the response to this excitation contains components at frequencies that are linear combinations of the excitation frequencies. The document provides for measuring a measuring voltage component at a frequency that corresponds to, for example, the frequency HF−BF and/or HF+BF. The component becomes proportional to the amount of superparamagnetic material arranged in the measuring coil, when a DC component is applied in addition. This amount reverses when the DC component is reversed, which makes it possible to improve the signal-to-noise ratio of the measurement, when an excitation sequence having a plurality of DC component values (in general having an average value of zero) is performed.

After having appropriately placed the analysis medium in (or close to) the measuring coil, a plurality of measurements of an electromotive force, taken off in the measuring circuit, are performed. The plurality of measurements is indexed by the separate DC current values injected into the measuring circuit. These measurements are then combined to form a measurement vector. The vector is itself combined with a signature vector of a standard magnetic mass, for estimating the mass of superparamagnetic material present within a measuring perimeter of the device. This combination can implement, in particular, a scalar product of the two vectors.

This prior art has several disadvantages.

When the analysis medium is formed of a column filled with a porous material, it is generally not possible to verify the correct performance of the test by way of a measurement in the region of a control zone. This lack of validation of the test is disadvantageous.

When a control zone of this kind exists, as may be the case for an analysis medium in the form of a strip that allows for the lateral migration of the sample, the use thereof requires two successive measurements to be made, one in the region of the capture zone 1c, and the other in the region of the control zone 1d. This duplication of the measurement requires the manual movement of the test strip relative to the measuring device, or for the device to be equipped with a movable test strip receptacle. In any case, this requirement makes it slower to obtain the result of the immunological test. Moreover, the measurement is very sensitive to the distance separating the measuring zone from the measuring coil, which requires very precise and repeatable control of the position of the strip.

When the measuring device is used to provide an estimation of the magnetic mass present in the capture zone 1c, and thus to estimate the amount of analytes A in the sample E, the result provided is sometimes imprecise. This is due to the fact that the amount of magnetically marked agents 2 initially present in the sample E is generally not well known. Some of these agents 2 may furthermore get caught in the migration zone and not reach the capture zone 1c and/or the control zone 1d. Moreover, the magnetic properties of the microbeads, as well as the migration phenomena, vary fairly significantly, depending on the temperature.

Consequently, the measurement of the absolute magnetic mass present in the capture zone 1c is information that cannot be used particularly reliably, in the solutions of the prior art, for estimating the amount or the concentration of analytes A in the sample E.

### BRIEF SUMMARY

An aim of the present disclosure is to overcome at least some of the above-mentioned disadvantages.

In order to achieve this aim, and according to a first aspect, the present disclosure proposes an analysis medium for immunoassay, comprising a substrate for promoting the natural or forced flow of a liquid sample likely to comprise analytes, the substrate comprising a capture zone.

According to the present disclosure, the capture zone comprises, immobilized on or in the substrate, a plurality of first capture agents capable of selectively retaining complexes bound to the analytes, and a plurality of second capture agents capable of selectively retaining reference agents.

According to other advantageous and non-limiting features of the present disclosure, taken individually or in any technically possible combination:
the substrate also comprises, upstream of the capture zone in the flow direction, a plurality of marked test agents of a first type of magnetic microbeads, the test agents being capable of binding to the analytes, and a plurality of marked reference agents of a second type of magnetic microbeads, the first type of magnetic microbeads and the second type of magnetic microbeads having different superparamagnetic properties;
the first capture agents are associated, respectively, with complexes comprising analytes and marked test agents of a first type of magnetic microbeads, and the second capture agents are associated with marked reference agents of a second type of magnetic microbeads, the first type of magnetic microbeads and the second type of magnetic microbeads having different superparamagnetic properties;
   the substrate is formed by a porous material;
   the substrate takes the form of a strip;
   the substrate takes the form of an amount of material arranged in a column.

According to another aspect, the present disclosure proposes a method for detecting the presence of analytes in a liquid sample, the method comprising the following steps:
bringing the sample together with marked test agents of a first type of magnetic microbeads, the test agents being capable of binding to the analytes, and marked reference agents of a second type of magnetic microbeads, the first and second types of magnetic microbeads having different superparamagnetic properties;
applying the sample to an analysis medium before or after the step of bringing together;
selectively retaining, on or in a capture zone of the analysis medium, at least some of the test agents bound to analytes, and reference agents, by means, respectively, of a plurality of first capture agents and a plurality of second capture agents;
successively exposing the capture zone to a plurality of excitation magnetic fields of different average intensities, measuring the combined magnetic response of the microbeads of the first type and of the microbeads of the second type, present in the capture zone, for each excitation magnetic field, and thus forming a measurement vector;
processing the measurement vector in order to determine the absolute or relative amount of microbeads of the first type and of microbeads of the second type.

According to other advantageous and non-limiting features of the present disclosure, taken individually or in any technically possible combination:
the combined magnetic response corresponds to the second derivative of the function linking the excitation field to the magnetic induction of the microbeads of the first type and the microbeads of the second type;
the step of processing the measurement vector comprises searching, in a standard vector base, for a standard vector that is closest to the measurement vector, each standard vector of the base being associated with a known amount of microbeads of the first type and of microbeads of the second type;
the step of processing the measurement vector comprises digitally searching for the respective magnetic masses of the nanoparticles that make up the microbeads of each type;
the step of processing comprises locating a first peak and a second peak of the magnetic response associated, respectively, with the microbeads of the first type and with the microbeads of the second type in order to determine the gradients linking the first peak and the second peak to the origin;
the step of processing comprises adjusting the combined magnetic response in order to make a second peak of this response, associated with the microbeads of the second type, correspond to a reference peak of the signature of the microbeads of the second type or standard vectors.

### BRIEF DESCRIPTION OF THE DRAWINGS

Other features and advantages of the present disclosure will become clear from the following detailed description of embodiments of the present disclosure, with reference to the accompanying drawings, in which:

FIGS. 1A and 1B show an analysis medium of the prior art, before and after use, respectively;

FIG. 2 shows the second derivative of the relationship linking an excitation magnetic field to the magnetic induction caused by the field in two superparamagnetic materials having different properties;

FIGS. 3A and 3B show an analysis medium according to an embodiment of the present disclosure, before and after use, respectively;

FIG. 4 shows the superparamagnetic characteristic of a capture zone 1c of an analysis medium 1 for samples E comprising increasing amounts of analytes A;

FIG. 5 shows the effect of the variability of the distance on the superparamagnetic characteristic identified by a measuring device.

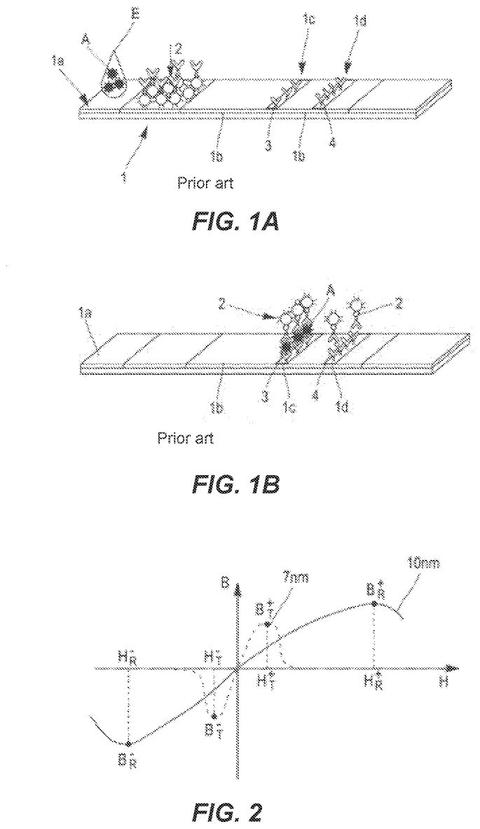

### DETAILED DESCRIPTION

In order to simplify the following description, the same reference signs are used for identical elements or elements performing the same function in the prior art or in the different embodiments of the present disclosure.

Microbeads of the first and of the second type.

In a general manner, the principles that underlie the present disclosure involve using two different types of microbeads on or in the analysis medium 1, i.e., microbeads having different superparamagnetic characteristics. The microbeads are referred to, respectively, as microbeads of the first type M1 and microbeads of the second type M2.

As has already been stated in the introduction of this disclosure, the microbeads of the first type M1 and of the second type M2 are formed of a binder that incorporates superparamagnetic nanoparticles. Each type of microbeads M1, M2 contains an amount of superparamagnetic nanoparticles that is fixed, from one microbead to another. The microbeads M1 of the first type are functionalized (for example, by antibodies selected so as to be capable of binding to the analytes A of the sample E that will undergo the test) and thus form test agents T. The microbeads of the second type M2 are, in turn, functionalized so as to prevent the binding (or, in a more general manner, to prevent any binding with any form of analyte, if a plurality of types of analytes are likely to be present in the sample E), and thus to form reference agents R. In other words, the reference agents R are thus incapable of forming complexes with the analytes.

The microbeads of the first type and of the second type M1, M2 have very specific superparamagnetic characteristics. “Superparamagnetic characteristic” means the relationship or the graph linking an excitation magnetic field H of these nanoparticles to the magnetic induction B in the material, caused by the field H, B=f(H), or to the first, second or third derivative of the relationship. A graph of this kind of the function f, the function itself (or one of its derivatives) forms, as it were, a unique signature of the superparamagnetic characteristic of a type of microbead.

According to an important aspect of the present description, the superparamagnetic characteristics of the first type M1 and of the second type M2 of microbeads are different from one another. In other words, exposed to the same excitation magnetic field, the magnetic induction of the nanoparticles of the microbeads of the first type M1 and of the second type M2 are different. The graphs or the functions linking, respectively, for these two types of microbeads, the excitation field H to the magnetic induction B, are different from one another.

Such a distinction in the superparamagnetic behavior can be achieved by selecting nanoparticles of different types, for each type of microbeads. The nanoparticles can be, in particular, different chemical compositions (for example, Fe₃O₄ particles in one case, and Fe₂₀O₁₈₀ in the other case). They may alternatively be of different sizes (for example, having a large diameter of 7 nm in one case, and of 10 nm in the other case). It is, of course, also possible to mix these properties of size and of chemical nature.

Thus, FIG. 2 shows, respectively, the respective superparamagnetic characteristics of two types of microbeads, a first type of microbead comprising nanoparticles of Fe₃O₄ of 7 nm diameter (in dotted lines) and the second type of microbeads having the same types of nanoparticles, but this time of 10 nm diameter (in a solid line). The curves shown are sensitivity curves, i.e., of the second derivative of the function f linking an excitation field H to the magnetic induction B of the nanoparticles.

It will be noted that, in FIG. 2, the superparamagnetic characteristics of the microbeads have the general appearance of a similar sinusoidal arc. They both pass through the reference center and exhibit magnetization extreme values B+, B− for field values H+, H−. However, the superparamagnetic characteristics of the two types of microbeads differ greatly from one another, in particular, by the level of the extreme values BR+, BR− (for the microbeads of the second type), BT+, BT− (for the microbeads of the first type) and by the values of the excitation field HR+, HR− (for the microbeads of the second type) and HT+, HT− (for the microbeads of the first type) to which the nanoparticles must be exposed in order to reach these extreme values.

For reasons that will become apparent in the remainder of this description, advantageously the magnetic microbeads M1 of the first type (which will magnetically mark the test agents T) will be selected such that their superparamagnetic characteristics develop over a relatively narrow range [H_{T}−, H_{T}+], and the magnetic microbeads of the second type M2 (forming the reference agent R) will be selected such that their superparamagnetic characteristics develop over a relatively wide period [H_{R}−, H_{R}+].

In a general manner, when the nanoparticles of the microbeads of the first and of the second type M1, M2 differ with respect to their size, it will be ensured that those having the largest diameter are of a size of at least 20% greater than those having the smallest diameter, and advantageously at least 30%, or indeed over 40%. It is thus ensured that the differences of the superparamagnetic characteristics between the two types of microbeads M1, M2 will be clearly marked.

When the superparamagnetic characteristic, shown in FIG. 2 in the form of continuous functions, is sampled in a plurality of values according to the X-axis and shown in the form of a vector, the vector will be referred to by the term “signature.”

### 1st Embodiment of the Analysis Medium—Test Strip

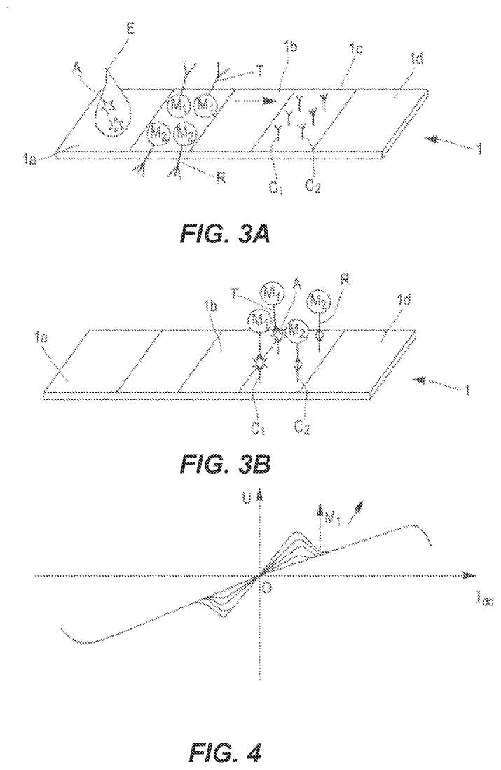

With reference to FIGS. 3A and 3B, an analysis medium 1 is shown, for application in the field of immunoassay, which makes it possible to implement the principles that have just been set out.

The analysis medium 1 is formed, in this embodiment, by a planar substrate, in this case in the form of a strip, for receiving the liquid sample E in the region of a receiving zone 1a, in this case arranged at an end of the substrate. The substrate 1 is made of a porous material that promotes the lateral flow of the solution, by capillary action, from the receiving zone 1a to the distal end of the strip 1.

In the example shown, the receiving zone 1a adjoins a zone on which a plurality of marked test agents T of a first type M1 of magnetic microbeads, and a plurality of marked reference agents R of a second type M2 of magnetic microbeads, are arranged.

The test agents T are capable of binding to the analytes A of the sample E, in the case, of course, of these analytes A indeed being present. This is not the case for the reference agents R. When a test agent T binds to an analyte A, they form an assembly that is referred to as a “complex” in the remainder of this disclosure, which is magnetically marked by a microbead M1 of the first type.

Whether or not they are bound to the analytes A, the test agents T, as well as the reference agents R, migrate laterally, carried along by the lateral flow phenomenon already mentioned, and progress through a migration zone 1b in order to reach a capture zone 1c arranged downstream of the analysis medium 1, in the flow direction.

As an alternative to the embodiment shown in these drawings, in which the test agents T and the reference agents R are arranged on the substrate 1 beside the receiving zone 1a, it is possible to provide for the agents to be placed directly on the receiving zone 1a or further downstream of the zone, close to the capture zone 1c.

It is also specified that it is not necessary for the analysis medium 1 to be originally provided with reference agents R and test agents T. These may be mixed with the sample E in advance, before the sample is poured onto the receiving zone 1a of the test strip 1.

Nonetheless, in any case care will be taken to bring the sample together with the test agents T and the reference agents R such that this is achieved before or after the analysis medium 1 is impregnated by the sample E.

The capture zone 1c of the analysis medium in turn comprises a plurality of first capture agents C1 and a plurality of second capture agents C2, the agents both being immobilized on the substrate.

The first capture agents C1 are capable of selectively retaining the complexes formed of analytes A and test agents T, i.e., magnetically marked by the microbeads M1 of the first type. The second capture agents C2 are in turn capable of selectively retaining the reference agents R, and thus marked by the microbeads M2 of the second type. “Selectively” means that these capture agents retain and immobilize only the agents for which they are capable of binding.

According to the present disclosure, the analysis medium may thus comprise just one capture zone 1c comprising, immobilized in a manner mixed together, both the plurality of first capture agents C1 and the plurality of second capture agents C2.

For the sake of completeness, it is specified that FIG. 3A shows an analysis medium 1 according to the present disclosure while the sample E is being poured onto the substrate. FIG. 3B in turn shows the same analysis medium 1 after the lateral flow of the sample E has caused the migration of the reference R and test T agents toward the capture zone 1c. It will be noted that the complexes are indeed retained in the region of the capture zone 1c by the first capture agents C1. The test agents T not bound to analytes A have continued their migrations toward a “trash” zone 1d of the medium 1. The reference agents R are in turn retained in the capture zone 1c by the second capture agents C2, so as to saturate the second agents C2 in the zone.

In all cases, such a configuration of the analysis medium makes it possible for the medium not to comprise a capture zone. The migration indicators are thus formed by the reference agents R marked by the microbeads M2 of the second type and detected in the region of the capture zone 1c, and not by the test agents T that are not bound to analytes and detected in the region of a control zone 1d, as in the prior art cited in the introduction of the present description. In this way, the use of the analysis medium no longer requires two successive measurements to be made, one in the region of the capture zone 1c and the other in the region of the control zone 1d, but a single measurement in the region of the capture zone 1c.

### 2nd Embodiment of the Analysis Medium—Test Column

According to an alternative embodiment that is not shown, the analysis medium 1 may take the form of a column that is open at each end and in which a porous material, forming a substrate, has been arranged. The substrate comprises, immobilized in at least a portion of its thickness in the column, the first and second capture agents C1, C2. The capture agents C1, C2 have the same properties as those described in relation to the first embodiment. The portion of the thickness of the substrate in which the agents are immobilized forms the capture zone 1c of the analysis medium 1 according to this embodiment. The capture zone 1c of the analysis medium 1 is thus in this case “volumetric,” in contrast with the “planar” capture zone of the first embodiment.

In a manner similar to that set out within the context of the first embodiment, the test T and reference R agents may be arranged in the column, upstream of the capture zone 1c, before the sample E is applied, or may be mixed with the sample E before the flow thereof is forced through the substrate that fills the column at least in part.

Whatever the way in which the sample E is brought together with the test T and reference agents R, it is desirable to promote the binding between the test agents T and the analytes A. The sample is then forced to flow through the column and the substrate, in order to cause the test agents T, the complexes and the reference agents R to migrate toward the capture zone 1c. The test agents T bound to the analytes, and the reference agents R, are selectively retained in the zone, by way of the capture agents C1, C2.

The portion of the porous substrate through which the sample E passes and that does not comprise a capture agent C1, C2 constitutes a migration zone 1b of the analysis medium 1, similar to that of the first embodiment. The test agents T not bound to analytes, and thus not retained in the capture zone 1c, are evacuated from the column together with the rest of the liquid sample E.

Irrespective of the embodiment selected for the analysis medium 1, and advantageously, the capture zone 1c is dimensioned so as to be entirely within the scope of the measuring perimeter of a measuring device. As will be set out in detail in the following, it is thus possible to perform, in a single step (i.e., without moving the analysis medium 1 with respect to the measuring device), a measurement of a signal intended for simultaneously determining the presence and/or the amount of microbeads of the first and of the second type M1, M2, i.e., the presence and/or the amount of complexes and of reference agents R.

Method for detecting the presence of analytes A in a sample E.

A method for detecting the presence of analytes A in a sample E, which benefits from an analysis medium 1 according to those that have just been set out, will now be set out. In a general manner, and as is well known in the field of immunoassay, it is desired to determine the amount of microbeads of the first type M1 associated with the test agents T, in order to infer therefrom the presence of analytes A. More specifically, the present disclosure seeks to determine the absolute or relative amount of microbeads of the first and of the second type M1, M2. Each type of microbeads M1, M2 contains an amount of superparamagnetic nanoparticles that is constant, from one microbead to another, such that determining the amount of microbeads of one type and/or of the other type is equivalent to determining the mass of nanoparticles present in all the microbeads of one type and/or of the other type.

As has been seen, this method comprises bringing the sample E together with test agents T magnetically marked by the microbeads of the first type M1 and with reference agents R magnetically marked by the microbeads of the second type M2. The microbeads of the first and of the second type M1, M2 comprise nanoparticles having different superparamagnetic characteristics. This step of bringing together, in particular, makes it possible to bind the test agents T to the analytes, if these are present in the sample E, in order to form complexes.

The method comprises, before or after the step of bringing together, applying the liquid sample E onto or into the analysis medium 1. The porous nature of the substrate forming this medium promotes the natural, or indeed forced, flow, depending on the nature of the medium, of the solution, and the migration of complexes, reference agents R and test agents T that are not bound, toward the capture zone 1c.

In the region of the capture zone 1c, the first capture agents C1 bind to the complexes that they selectively retain in this zone. The non-bound test agents T are not retained by the first capture agents C1, nor indeed by the second capture agents C2 immobilized in the capture zone 1c. They thus continue their migrations toward the trash zone 1d of the strip 1 or are evacuated from the column.

Simultaneously, the second capture agents C2 selectively retain the reference agents R in the capture zone 1c, i.e., only the reference agents R are retained by the second capture agents C2.

Advantageously, it is desirable to arrange, in the capture zone 1c, an amount that is relative constant from one immunological test to another. This can be achieved by integrating a controlled amount of reference agents R and of second capture agents C2 into the analysis medium.

At the end of the migration and selective retention step, a measurement is performed that aims to obtain the combined superparamagnetic characteristic of the microbeads of the first type M1 (associated with the complexes) and the microbeads of the second type M2 (associated with the reference agents R) arranged in the capture zone 1c.

In order to achieve this, it is possible to use a measuring device of the type described in the European document cited in the introduction to this disclosure. The device makes it possible to estimate, in the form of a measurement vector, the second derivative of the relationship f linking the excitation magnetic field H and the magnetic induction B in the superparamagnetic material. It is possible to use other measuring techniques for estimating the function f itself (rather than the second derivative thereof) or for estimating the first or third derivative thereof. However, establishing the sensitivity function (the second derivative of the function f linking an excitation field H to the magnetic induction B) by way of the measuring device constitutes the preferred embodiment.

In order to implement the measuring step, and whatever the measuring device used, the analysis medium 1 is arranged in the measuring device such that the capture zone 1c is located in a measuring coil of the device, or close to a coil of this kind, and within the measuring perimeter of the device, in order to be able to be entirely exposed to the magnetic fields produced.

The capture zone 1c is exposed to an excitation magnetic field having a first frequency (for example, a relatively low frequency), a second frequency that is different from the first (for example, a relatively high frequency), and also having a substantially constant component that makes it possible to fix the average intensity of the field. The exposure is repeated successively for the different constant components of the excitation field, so as to expose the capture zone to a plurality of excitation fields of varied and different average intensities.

In practice, the excitation fields are controlled by the intensity and the frequency of currents at the first and at the second frequency, and by a constant current injected into a measuring circuit (comprising the measuring coil) of the measuring device.

Upon each successive exposure, a voltage (an electromotive force) is taken off at measuring terminals of the measuring circuit, which voltage is processed in order to extract therefrom a component at a mixing frequency, the mixing frequency being a linear combination of the first and second frequencies of the excitation field. The value of this component is linked (proportional) to the amount of superparamagnetic material present in the field H produced by the measuring coil, i.e., to the respective masses of the superparamagnetic nanoparticles present in the microbeads of the first and second types. In other words, upon each successive exposure, the combined magnetic response of the microbeads of the first type M1 and of the microbeads of the second type M2, present in the capture zone 1c, for each excitation magnetic field H, is measured.

By repeating this processing for each of the exposure steps, it is possible to form a measurement vector that samples, for different values of DC current (representative of an excitation field), the combined superparamagnetic characteristic of the microbeads of the first and of the second type M1, M2 present in the capture zone 1c.

By way of example of superparamagnetic characteristics that can be identified by this approach, FIG. 4 shows the superparamagnetic characteristic of a capture zone 1c of an analysis medium 1 for samples E comprising increasing amounts of analytes E (and thus of magnetic microbeads of the first type M1). The X-axis of the graph corresponds to the DC current Idc injected into the measuring circuit of the measuring device. It represents the average intensity of the excitation field H to which the capture zone 1c has been exposed during the measurement. The Y-axis corresponds to the voltage component U taken off by the measuring circuit at the mixing frequency. This measurement, at each given value of the DC current Idc, is proportional to the amount of magnetic mass present in the capture zone 1c. In this example, the microbeads of the first type M1 comprise nanoparticles of Fe3O4 of 7 nm diameter, and the second type of microbeads M2 comprise the same types of nanoparticles, of 10 nm diameter. The sensitivity functions (the function f″) of the microbeads are those shown in FIG. 2, on which comments have already been made.

It is noted, in this representation, that, for a given amount of analytes and microbeads of the first type M1, the superparamagnetic characteristic of the capture zone combines the superparamagnetic of the microbeads of the first type M1 and the superparamagnetic characteristic of the microbeads of the second type M2, in their respective proportions. The greater the number of microbeads of the second type M2, the more the superparamagnetic characteristic associated with these microbeads is marked in the combined characteristic. The interest in selecting the distinct nature of the microbeads of the first and second type M1, M2, and in ensuring that the extreme values of the component taken off appear for currents Idc in different value ranges, can thus be understood. It is thus possible to easily separate the contribution of each type of microbeads to the combined superparamagnetic characteristic.

In any case, at the end of the measuring step of a method according to the present disclosure, a measurement vector Vm is present that is representative of the combined superparamagnetic characteristic of the microbeads of the first and of the second type M1, M2 arranged in the measuring perimeter of the measuring device (and thus in the capture zone 1c).

In a following step, the measurement vector Vm is used for determining the absolute or relative amount of microbeads of the first type M1 and of microbeads of the second type M2.

According to a first approach, the measurement vector Vm is used by comparing it to a standard vector base Vij, a standard index vector i, j having been obtained by way of the measuring device for a known amount of Ni microbeads of the first type M1 and of Nj microbeads of the second type M2, arranged within the measuring perimeter. The comparison thus seeks to identify the standard vector that is closest to the measurement vector Vm, i.e., to identify the indices i*, j* that minimize the norm (Vm−Vij) for each pair (i,j) indexing the standard vector base. The amounts of microbeads of the first type M1 and of microbeads of the second type M2 are thus provided, respectively, by the amounts Ni* and Nj* associated with the vector Vi*j* in the vector base.

According to another approach, it is possible to digitally search for the respective amounts of microbeads, by positing that the measurement vector Vm is formed of the sum, respectively weighted by the magnetic masses m1, m2 of the nanoparticles making up the microbeads of each type, of the signatures S1, S2 of each type of microbead M1, M2:

Vm=m1*S1+m2*S2.

Applying a decorrelation/separation of sources makes it possible to simultaneously determine the mass m1 and the mass m2, and to easily obtain the respective amounts of microbeads of each type. It may thus be a case of implementing a digital optimization technique aiming to determine the magnetic masses m1, m2 such that the norm (Vm−m1*S1−m2*S2) is minimal.

Both of these approaches have the advantage of providing an absolute amount of microbeads of the first type M1 and of microbeads of the second type M2. The amount of microbeads of the second type M2 (associated, for the record, with a reference agent R) may be used, if it exceeds a predetermined threshold, as an indicator of the correct migration of this agent R, and thus to confirm the smooth running of the immunological test. The amount of microbeads of the first type M1 (associated, for the record, with the test agents T) may make it possible to quantify the presence of the analyte A in the sample E.

In an advantageous manner, it is also possible to calculate the ratio between the two amounts of microbeads, and thus obtain a result independent of the starting amounts of these microbeads, or indeed of the influence of other parameters (such as the temperature) on the migration of the agents R, T and on their superparamagnetic properties. It is noted, in particular, that a minority of reference agents R, test agents T, or complexes, may remain caught, during the migration, in the migration zone 1b. The possibility of this zone comprising amounts of these agents R, T and complexes, which can reasonably be assumed to be identical, thus cannot be excluded. Consequently, the absolute amounts of microbeads of the first and of the second type M1, M2 retained in the capture zone 1c may vary from one test to another. Providing a relative value, in the form of the ratio proposed above, makes it possible to mask this phenomenon of catching, and to provide a more reliable result of the immunological test.

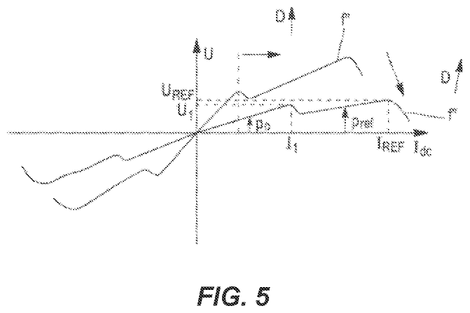

According to an advantageous variant of the present disclosure, it is desirable to compensate a possible variability in the distance separating the capture zone 1c, on or in which the microbeads M1, M2 are arranged, and the measuring coil that generates, in particular, the excitation field at the origin of the measurement. FIG. 5 shows the effect of this variability on the superparamagnetic characteristic identified by the measuring device.

Although the relationship linking the current injected into the measuring circuit of the measuring device, to the excitation field generated, is well established, the relative position of the capture zone with respect to the field may sometimes be less well controlled, and the intensity of the field in the region of the capture zone 1c may vary from one measurement to another. The variation of the sensitivity, identified in the course of a variation of the distance D, can be seen in FIG. 5, because the general gradient of the function f″ reduces as the distance D increases. The variation of the position of the extreme values (the current required for saturating the nanoparticles having to be increased when the particles are further away) is also observed.

However, complementary studies have demonstrated that, in turn, the ratio of the gradients p0/pref remained relatively invariant with the distance D.

The gradient p0 corresponds to the ratio U1/I1, where I1 is the current injected into the measuring circuit that makes it possible to achieve the voltage peak U1 associated with the superparamagnetic characteristic of the microbeads of the first type M1. It is thus the gradient linking the origin to a first peak of the combined magnetic response, the first peak being associated with the microbeads of the first type M1.

The gradient pref corresponds to the ratio Uref/Iref, where Iref is the current injected into the measuring circuit that makes it possible to achieve the voltage peak Uref associated with the superparamagnetic characteristic of the microbeads of the second type M2. It is thus the gradient linking the origin to a second peak of the combined magnetic response, to the microbeads of the second type M2 at the origin.

Thus, according to another advantageous approach, the measurement vector Vm is processed so as to locate the maximum values U1 and Uref, as well as the currents I1 and Iref for which these maximum values are obtained, in order to determine the gradients p0 and pref. If the maximum value U1 cannot be identified (case where the amount of microbeads of the first type M1 is zero), by default p0=pref is taken. At the end of the processing, it is thus possible to provide the relative amount of microbeads of the first type M1 vs microbeads of the second type M2 by returning, for example, the ratio r=p0/pref−1, even when the distance separating the microbeads of the measuring device is poorly controlled. Advantageously, in order to perform this analysis, it is possible to reconstruct the sensitivity function f″ by interpolation of the measuring points that form the measurement vector Vm.

It is possible to use the same principles underlying the preceding approach in order to adjust the measuring vector, the standard vectors, and the signatures provided by the measuring device, in order to make these vectors more independent of the distance.

Supposing that the amount of microbeads of the second type M2 (associated with the reference agents R retained in the capture zone 1c) is substantially constant, it is possible to determine the adjustment factors fx, fy to be applied, respectively, according to the X-axis (current Idc) and according to the Y-axis (measured voltage U) on the graph representing the measurement vector, in order to cause the maximum values (Iref, Uref) of the measurement vector to coincide with the maximum values of the signature vector S2 of the microbeads of the second type M2. The processing makes it possible to prepare an adjusted measurement vector that can be used according to one of the first two approaches set out above, in order to determine the absolute amount of microbeads of the first type M1 (associated with the test agents T). Once again, this processing may require reconstruction of the sensitivity function f″ by interpolation of the measuring points that form the measurement vector Vm. It is thus possible to reconstruct an adjusted measurement vector, having a standard cardinality.

Of course, the present disclosure is not limited to the embodiments described, and it is possible to add variants thereto, without extending beyond the scope of the invention as defined by the claims.

Although an application of the measurement method in the field of immunoassay has been set out here, the measurement method may be applied more generally for simultaneously measuring the magnetic masses of a plurality of first and a plurality of second superparamagnetic nanoparticles having different characteristics. This method implements a set aiming to successively expose the first and second nanoparticles to a plurality of magnetic fields of different average intensities, measure their combined magnetic responses, and form a measurement vector. It then implements a step aiming to process the measurement vector in order to determine the magnetic masses of the plurality of first and second superparamagnetic nanoparticles.

1. An analysis medium for immunoassay, comprising: a substrate for promoting the natural or forced flow of a liquid sample likely to comprise analytes the substrate having a capture zone, the capture zone comprising, immobilized on or in the substrate, a plurality of first capture agents capable of selectively retaining complexes bound to the analytes and a plurality of second capture agents capable of selectively retaining reference agents that are functionalized so as to prevent binding to the analytes, so as to make it possible to perform, in a single step, a measurement of a signal, intended to simultaneously determine the presence and/or the concentration of complexes and reference agents.

2. The analysis medium of claim 1, wherein the substrate also comprises, upstream of the capture zone in the flow direction, a plurality of marked test agents of a first type of magnetic microbeads, the test agents being capable of binding to the analytes and a plurality of marked reference agents of a second type of magnetic microbeads, the first type of magnetic microbeads and the second type of magnetic microbeads having different superparamagnetic properties.

3. The analysis medium of claim 1, wherein the first capture agents are associated, respectively, with complexes comprising analytes and marked test agents of a first type of magnetic microbeads, and the second capture agents are associated with marked reference agents of a second type of magnetic microbeads, the first type of magnetic microbeads and the second type of magnetic microbeads having different superparamagnetic properties.

4. The analysis medium of claim 1, wherein the substrate comprises a porous material.

5. The analysis medium of claim 1, wherein the substrate comprises a strip.

6. The analysis medium of claim 1, wherein the substrate comprises an amount of material arranged in a column.

7. A method for detecting the presence of analytes in a liquid sample, the method comprising:
bringing the sample together with marked test agents of a first type of magnetic microbeads, the test agents being capable of binding to the analytes, and marked reference agents of a second type of magnetic microbeads, the first and second types of magnetic microbeads having different superparamagnetic characteristics;
applying the sample to an analysis medium before or after the bringing of the sample together with the marked test agents and the marked reference agents;
selectively retaining, on or in a capture zone of the analysis medium, at least some of the test agents bound to the analytes, and reference agents, by way of, respectively, a plurality of first capture agents and a plurality of second capture agents;
successively exposing the capture zone to a plurality of excitation magnetic fields of different average intensities, measuring a combined magnetic response of the microbeads of the first type and of the microbeads of the second type, present in the capture zone, for each excitation magnetic field, and thus forming a measurement vector; and
processing the measurement vector to determine the absolute or relative amount of microbeads of the first type and of microbeads of the second type.

8. The method of claim 7, wherein the combined magnetic response corresponds to a second derivative of the function linking the excitation field to the magnetic induction of the microbeads of the first type and the microbeads of the second type.

9. The method of claim 7, wherein the processing of the measurement vector comprises searching, in a standard vector base, for a standard vector that is closest to the measurement vector, each standard vector of the base being associated with a known amount of microbeads of the first type and of microbeads of the second type.

10. The method of claim 7, wherein the processing of the measurement vector comprises digitally searching for respective magnetic masses of nanoparticles that make up the microbeads of each type.

11. The method of claim 7, wherein the processing of the measurement vector comprises locating a first peak and a second peak of the magnetic response associated, respectively, with the microbeads of the first type and with the microbeads of the second type to determine the gradients linking the first peak and the second peak to the origin.

12. The method of claim 7, wherein the processing of the measurement vector comprises adjusting the combined magnetic response to make a second peak of this response, associated with the microbeads of the second type, correspond to a reference peak of the signature of the microbeads of the second type or standard vectors.

13. The method of claim 8, wherein the processing of the measurement vector comprises searching, in a standard vector base, for a standard vector that is closest to the measurement vector, each standard vector of the base being associated with a known amount of microbeads of the first type and of microbeads of the second type.

14. The method of claim 8, wherein the processing of the measurement vector comprises digitally searching for respective magnetic masses of nanoparticles that make up the microbeads of each type.

15. The method of claim 8, wherein the processing of the measurement vector comprises locating a first peak and a second peak of the magnetic response associated, respectively, with the microbeads of the first type and with the microbeads of the second type to determine the gradients linking the first peak and the second peak to the origin.

16. The analysis medium of claim 2, wherein the substrate comprises a porous material.

17. The analysis medium of claim 2, wherein the substrate comprises a strip.

18. The analysis medium of claim 3, wherein the substrate comprises a porous material.

19. The analysis medium of claim 3, wherein the substrate comprises a strip.
